(19) United States
(12) Patent Application Publication
(10) Pub. No.: US 2002/0198552 A1
(43) Pub. Date: Dec. 26, 2002

(54) Intraoral hygiene device

(76) Inventor: Edward Yavitz, Rockford, IL (US)

Correspondence Address: GEORGE R CORRIGAN, 5 BRIARCLIFF COURT, APPLETON, WI, 54915, US

(21) Appl. No.: 09/681,897

(22) Filed: Jun. 21, 2001

(51) Int. Cl. A61B 17/24

(52) U.S. Cl. 606/161

(57) ABSTRACT

A method and apparatus for treating breath includes an intraoral a pad is conformed to fit a palate of a human mouth. The pad has a first surface that is configured to lie along the palate of the human mouth. It also has an exterior surface exposed to the tongue of the human mouth. The exterior surface is at least partially abrasive. The pad preferably includes a bio-adhesive on the first surface and a predetermined dosage of medication is embedded in the pad. The exterior surface is preferably a water-insoluble material, such as a silicate, polypropylene, a wax, a natural polymer and a synthetic polymers in other embodiments. A self-supporting layer is provided between the exterior surface and the first surface.

### FIELD OF THE INVENTION

The present invention relates generally to the art of intraoral devices. More specifically, it relates to an intraoral device that removes food debris from the tongue and/or releases antiseptic, antioxidant and/or deodorizing compounds, thus promoting oral hygiene.

### BACKGROUND OF THE INVENTION

Eating inevitably leaves a residue of food and drink on the surface of the tongue, teeth and gums. This residue is a source of cavity and odor causing bacteria. It is estimated that 90% of mouth odor is caused by bacterial growth in the food debris trapped between the papillae ridges of the upper (dorsal) surface of the tongue. Mouth odor is embarrassing at the least, and may diminish personal interactions.

There are a number of common products used to reduce food debris (and thus combat bad breath), including mouthwash, tongue scrapers, toothpaste and toothbrushes. These have significant drawbacks. First their effectiveness is not always satisfactory. Second, it is not polite to use them in public.

One common type of prior art mouthwash is described in U.S. Pat. No. 4,289,755, and utilizes alcohol, sometimes in combination with herbs such as cinnamon and zinc, fluoride and/or citric acid which will temporarily kill bacteria, denature residual food debris and reduce odor. Unfortunately mouthwashes do not physically scrape or remove debris from between the papillae on the surface of the tongue. Thus, odor-causing bacterial growth commences within minutes after using these devices. Furthermore, gargling is not appropriate in public and the mouthwash must be expelled after use.

Breath mints or chewing gum have also been used to fight mouth odor, such as that shown in U.S. Pat. No. 4,409,202. Unfortunately, they are often ineffective, extremely temporary, easily noticed when being used, and can adversely affect the user's speech.

A two-sided tongue scraper is shown in U.S. Pat. No. 6,083,235 and includes a hard plastic loop for scraping the surface of the tongue on one end, and an applicator pad containing ingredients to prepare the tongue for scraping on the other end. This requires a two-stage procedure, which should not be performed in public. The flat scraper is supposed to reduce the gag reflex, but it likely cannot avoid the gag reflex in some users because it can easily be placed inside the mouth far enough along the tongue to elicit a gag reflex in any user. Indeed, the patent teaches to use an astringent and/or anesthetizing agents to further reduce the gag reflex, presumably because the shape of the flat scraper alone doesn't satisfactorily reduce the gag reflex. Additionally, it does not remove food between teeth or in the gum line.

Another tongue scraper is shown in U.S. Pat. No. 5,779,654. Again, a tongue scraper should not be used in public, does not prevent an unpleasant gag reflex if placed too far into the mouth, provides at best temporary relief since odor causing bacterial growth begins the moment scraping and treatment of the tongue surface stops, and does not remove food between teeth or in the gum line.

U.S. Pat. No. 5,181,505 teaches an indwelling oral pad with antiseptic, antibiotic characteristics; however, it requires an endotracheal tube to secure it within the mouth which would be an impediment to speech and social interaction.

The present inventor has previously invented intraoral devices (U.S. Pat. Nos. 5,462,049 and 5,638,810, hereby incorporated by reference) which have deodorant, antioxidant and antiseptic elements in enteric coated form for sustained temporal release inside the mouth. While these devices adequately reduce existing mouth odor, they do not attack the source of mouth odor, i.e., remove food residue trapped between the papillae ridges on the surface of the tongue, in the gums and on the teeth.

U.S. Pat. No. 5,578,315 provides for a mucosal adhesive device to deliver multiple physician prescribed pharmaceuticals in the oral cavity, but does not provide for remove food residue trapped between the papillae ridges on the surface of the tongue, in the gums and on the teeth, such as by tongue scraping. Furthermore, the antimicrobial and anti-inflammatory agents released are expensive, require medical supervision and have potentially adverse side effects.

Accordingly, there is a need for a device that removes food debris from, and treat the surfaces of the tongue, teeth and gums with antiseptic, antioxidant and/or breath fresheners, but does not require reintroduction into the mouth for each treatment. Preferably, the use of it would not be visible to others, so that the user could scrape their tongue of food debris and treat their mouth while in public. Also, it would preferably not cause a gag reflex or interfere with speech so that the user could engage in conversation while using it. Another preferred feature would be that the treatment lasts over an extended period of time, such as hours rather than minutes. Furthermore, it is known that saliva reduces the amount of residual food debris on the surface of teeth and gums. Thus, such a device would preferably induce saliva over an extended period of time, such as by the release of flavorings which provoke the reflex release of saliva.

### BRIEF SUMMARY OF THE INVENTION

According to a first aspect of the invention an intraoral device includes a pad that is conformed to fit a palate of a human mouth. The pad has a first surface that is configured to lie along the palate of the human mouth. It also has an exterior surface exposed to the tongue of the human mouth. The exterior surface is at least partially abrasive.

According to a second aspect of the invention a method of treating a mouth over a period of time includes forming a pad having a first surface configured to lie along the palate of the human mouth and providing an exterior surface of the pad that is at least partially abrasive.

According to a third aspect of the invention a method of treating a mouth over a period of time includes placing a pad in the mouth, adhering a first surface to the palate of the mouth, and scraping the tongue against an exterior surface of the pad that is at least partially abrasive.

The pad includes a bio-adhesive on the first surface, which may be one of natural gums, synthetic polymers, mucoadhesive polymers, hydrophillic polymers, natural polymers, saccharine derivatives, and cellulose derivatives, polyacrylate, polyacrylic acid, a carboxyvinyl polymer, a derivative of a carboxyvinyl polymer, pectin, alginate, karaya gum, pectin gum, xanthan gum, and guar gum in various alternative embodiments.

A predetermined dosage of medication is embedded in the pad in another embodiment. The medication may be provided as particles, perhaps enteric coated, and distributed through at least a portion of the pad. The medication is a flavored substance such as menthol, menthyl lactate, peppermint oil, paramenthane and caboxyamides, or the medication is an anesthetic, analgesic, antibiotic, anti-inflammatory, anti-bacterial, antimicrobial, antifungal, antihistamine, steroid and anti-neoplastic agent in various embodiments.

The exterior surface is a water-insoluble material, such as a silicate, polypropylene, a wax, a natural polymer and a synthetic polymers in other embodiments.

A self-supporting layer between the exterior surface and the first surface, such as one made of polyester, polypropylene, nylon, rayon, cellulose acetate, cellulose derivatives, cloth, paper, wax, synthetic fibers and natural fibers, that may be spray-bonded, spun-bonded, carded, thermal bonded, melt-blown, needled, wet-laid and dry-laid, is provided in various embodiments.

The pad includes an airborne contaminant absorber and the exterior surface is absorbent in another embodiment.

According to a fourth aspect of the invention an intraoral device for providing medication to a user includes a pad conformed to fit the palate of a human mouth. The pad includes an airborne contaminant absorber and a first surface configured to lie along the palate of a human mouth. The first surface has a bio-adhesive disposed thereon. An exterior absorbent surface of the pad is exposed to the tongue of a human mouth and a predetermined dosage of medication is embedded in the pad. The predetermined dosage of medication includes a plurality of particles distributed through at least a portion of the pad and some of the particles have enteric coatings exposed to the tongue of the wearer.

According to yet another aspect of the invention an intraoral device includes a pad having a first surface with a bio-adhesive disposed thereon and an exterior surface exposed to the tongue of a human mouth. The exterior surface is at least partially abrasive.

Other principal features and advantages of the invention will become apparent to those skilled in the art upon review of the following drawings, the detailed description and the appended claims.

### BRIEF DESCRIPTION OF THE SEVERAL VIEWS OF THE DRAWINGS

FIG. 1 is a top plan view of an intraoral device in accordance with the present invention;

FIG. 2 is a front elevation view of an intraoral device in accordance with the present invention;

FIG. 3 is a cross sectional view taken generally along line 3-3 of FIG. 1; and

FIG. 4 is a cross sectional view of an intraoral device in accordance with the invention.

Before explaining at least one embodiment of the invention in detail it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting. Like reference numerals are used to indicate like components.

### DETAILED DESCRIPTION OF THE INVENTION

While the present invention will be illustrated with reference to a particular embodiment implemented with particular materials, it should be understood at the outset that the invention may also be implemented with other embodiments using other materials.

Generally, the invention includes an intraoral device that is a pad, preferably adhered to the upper surface (palate) of the mouth using a bio-adhesive. The surface of the pad exposed to the tongue may include rough particles that scrape the tongue. Also, the pad may include medicine, preferably enteric coated particles, that is released into the mouth over time. These features serve to prevent and treat bad breath. Intraoral device, as used herein, includes a device used in the oral cavity over an extended period of time. Medication, as used herein, includes drugs, flavorants, and/or substances that have physiological effects psychological effects, or can be tasted or smelled.

Preferably, the device is a flat multi-layer or woven matrix pad stored in a dry state. When wet with saliva or water, an adhesive component is activated and the device adheres to the tongue, palate or to the mucosa of the mouth. The device has enteric coated ingredients including flavorings, antioxidants, medications, antibiotics or other therapeutic actives suitable for mucosal administration. Therapeutic actives would include anesthetic, analgesic, antibiotic, anti-inflammatory, antibacterial, antifungal, antihistamine, insulin, peroxide, cinnamon derivatives, steroid and antineoplastic agents. Flavored substance, as used herein, includes natural and artificial flavors, and substances that can be tasted or smelled.

Also included throughout the pad, and particularly in the outermost layer facing the upper surface of the tongue, are abrasive components consisting of water-insoluble materials selected from the group including, silicates, polypropylene, wax, natural or synthetic polymers or combinations thereof.

Preferably, the user places the pad on the upper hard palate of the mouth. Some users may allow the adhesive to melt and stick the device to the tip of the tongue, then rub the device with the tongue against all surfaces of the teeth in order to remove plaque and food debris. The device can then be placed using the tongue against the roof of the mouth where it will remain until physically removed. During that time, the flavorings and therapeutic ingredients will release as their enteric coatings melt over a prolonged period of time.

An intraoral device in accordance with the present invention is shown in FIGS. 1-4. Intraoral device 10 is designed to fit within the mouth of a wearer and to reduce, prevent or treat bad breath. Referring generally to FIG. 1, intraoral device 10 includes a flexible pad 12 configured for insertion into the mouth of a wearer. Flexible pad 12 may be designed for insertion into a variety of locations within the wearer's mouth, including disposition between the teeth and cheeks i.e. the buccal vestibule area of the human mouth. However, the pad is preferably configured to lie generally longitudinally along the roof of the mouth.

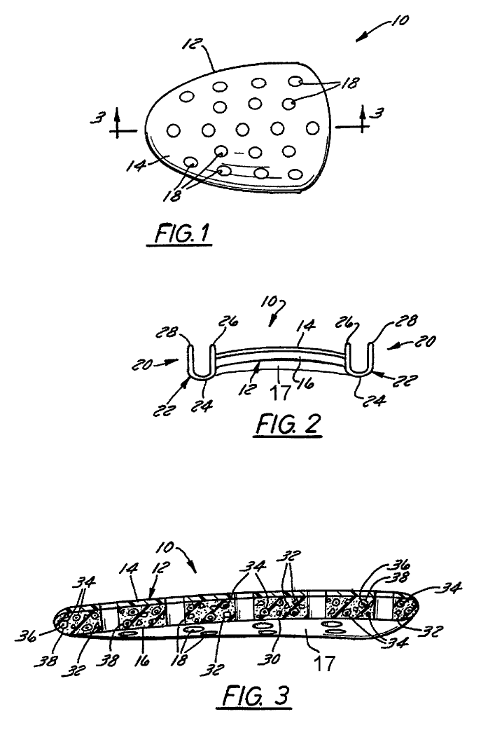

Pad 12 preferably includes an upper layer 14 (see FIG. 2) which engages or lies along the palate of the wearer. Upper layer 14 preferably includes or is a bio-adhesive so that the pad bio-adheres to the palate. Bio-adhering, as used herein, includes adhering using a bio-adhesive. Bio-adhesive, as used herein, includes an adherent that is not dangerous to be used internally, ingested, or in contact with skin or mucosal surfaces.

Preferably, pad 12 is configured to lie along the palate of the human mouth, and conformed to fit the palate of the mouth, such that it does not interfere with normal speech, breathing, and swallowing, etc. Configured to lie along the palate of the human mouth, as used herein, includes shaped consistent with, or flexible enough to adapt to, the contours of a palate. Conformed to fit a palate of a human mouth, as used herein, includes closely fitted to the palate, such that fluids do not easily pass over the palate.

The bio-adhesive should preferably be a substance that develops adhesion when coming into contact with the mucosa, such as a cellulose, a cellulose derivative, a polyacrylate, polyacrylic acid, a carboxyvinyl polymer, a derivative of a carboxyvinyl polymer, a lectin or natural material such as alginate derived from seaweed, natural gums selected from the group consisting of karaya gum, pectin gum, xanthan gum, guar gum, synthetic polymers, natural polymers, saccharide derivatives, or mixtures thereof.

Upper layer 14 may also generally be a non-absorbing material, such as a non absorbing plastic, to prevent prolonged contact between the other layers of flexible pad 12 (which includes abrasives) and the surface of the wearer's mouth.

Pad 12 also includes a self-supporting layer 16, which preferably lies adjacent upper layer 14 and is attached to upper layer 14. Self-supporting layer, as used herein, includes, a layer of a pad having sufficient strength to hold the shape of the pad. Layer 16 is provides support and shape to pad 12, and is preferably non-adhesive. Self-supporting layer 16 is made from one or more of polyester, polypropylene, nylon, rayon, cellulose acetate, cellulose derivatives, cloth, paper, wax, synthetic or natural fibers and mixtures thereof in various embodiments. Alternatives provide for the omission of layer 16.

Layer 16 may be formed by a process selected from the group consisting of spray-bonded, spun-bonded, carded, thermal bonded, melt-blown, needled, wet-laid, dry-laid and combinations thereof.

Adjacent layer 16 is an abrasive layer 17, which is exposed to the tongue of the wearer. Abrasive, as used herein, includes a surface that is sufficiently rough for tongue scraping or to effectively scrape a tongue. Tongue scraping, as used herein, includes scraping the tongue such that food debris trapped between the papillae ridges of the upper (dorsal) surface of the tongue is removed. Layer 16, in use, provides for a continual removal of food debris from the tongue.

A plurality of abrasive components 18 are shown in FIGS. 1 and 3 in layer 17, but may also be in layer 16. The abrasive components cause the pad to act as a tongue scraper, that is left in the mouth for an extended period of time. Also, it does not affect speech or swallowing.

In one embodiment layer 16 and 14 are combined, so that the abrasive particle are distributed throughout pad 12, or at least throughout a portion of pad 12. Distributed through at least a portion of the pad, as used herein, includes a distribution that may be equal or unequal over the portion of the pad. The adhesive components are preferably water-insoluble materials selected from the group including, silicates, polypropylene, wax, natural or synthetic polymers or combinations thereof.

Pad 12 may also have medication embedded in the pad. Embedded in the pad, as used herein, includes distributed though at least a portion of the pad, or disposed within the pad. The medication is, in various embodiments, one or more of menthols, menthyl lactates, peppermint oils, paramenthanes and caboxyamides, anesthetics, analgesics, antibiotics, anti-inflammatories, anti-bacterials, antimicrobials, antifungals, antihistamines, steroid sand anti-neoplastic agents.

In another embodiment (shown in FIG. 2) intraoral device 10 includes a retainer portion 20 attached to flexible pad 12. Retainer portion 20 may have various configurations to hold flexible pad 12 between the wearer's teeth, along side the wearer's teeth, or over the wearer's teeth.

Flexible pad 12 is, in one embodiment, at least partially impregnated with medication, as described above, The medication 34, may be seen in FIG. 3. In the illustrated embodiment, medication 34 is a breath freshener and encapsulated by enteric coatings 38. Some of breath freshener 34 provides instantaneous freshening once intraoral device 10 is placed in the wearer's mouth. Other breath freshener is encapsulated by enteric coatings 38 that are dissolved over time by the saliva of the wearer to provide a later release of medication. By encapsulating medication 34 with enteric coatings of different thicknesses, the release of breath freshener 34 is maintained over a longer period of time.

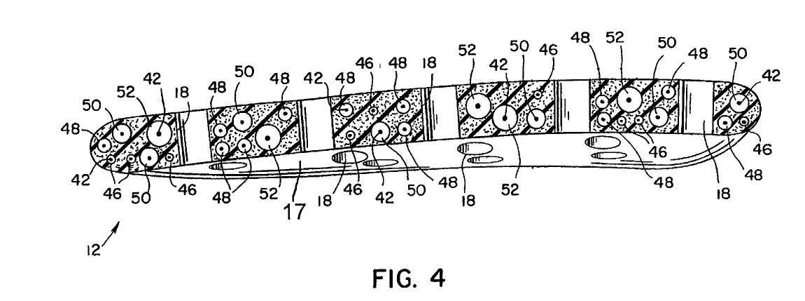

Flexible pad 12 may also be at least partially impregnated with a different medicinal substance 40 in place of or in combination with breath freshening substance 34 and abrasive material 18. (See FIG. 4.) In this embodiment, it may not be necessary, although still preferred, to include upper layer 14. Also, for some applications, the flexible pad should be configured to fit between the wearer's teeth and cheek. An airborne contaminant absorber can also be included (shown also as 18, such as in U.S. Pat. No. 5,638,810.

In this embodiment, flexible pad 12 includes a predetermined dosage of one or more medicinal substances 40. Preferably, the medicinal substance or substances 40 are divided into a plurality of portions or particles 42 that are distributed throughout at least part of flexible pad 12. Particles 42 dissolve within the mouth of the wearer to provide medication to the wearer over a sustained period of time. The length of this period can be controlled by placement of the particles or by creating particles of different sizes; however, it is preferred that the particles or portions 42 are coated with enteric coatings 44 that are dissolved by saliva within the wearer's mouth to release the medicine therein. By coating different particles with enteric coatings of different thicknesses, the rate of release of the medication can be controlled over a substantial period of time.

For example, some of the portions or particles 42 may be covered with a relatively thin enteric coating 46. Other portions 42 may be covered with a slightly thicker enteric coating 48. Still other portions 42 may be covered with an enteric coating 50 having a third thickness that differs from the enteric coatings 46 and 48. Still other portions 42 may have enteric coatings 52 that are quite thick to provide for a substantial delay prior to the release of medication from the encapsulated portions 42. The portions 42 can also be covered with a much greater range of enteric coatings with different thicknesses, depending on the type of medication, the need for consistency of release, and the length of time over which it is to be released.

A variety of medications can be administered to the individual wearing flexible pad 12, and the following list should not be considered inclusive. However, examples of medications, in addition to those described above, that can be administered via flexible pad 12 include viscous xylocaine, antiseptic agents, flouride, continuous antiacid, various antibiotics, nitroglycerin and acetazolamide.

In one embodiment the abrasive component is distributed through-out the pad, and may also be represented by particles 48.

In another embodiment the bio-adhesive, medication and abrasive components are dispersed throughout the self supporting layer. A variation provides that the adhesive and/or abrasive components are brought to the surface by chewing or an outer layer dissolving. This embodiment allows the user to adhere the device to any mucosal surface within the oral cavity and not worry about which side is up.

Numerous modifications may be made to the present invention which still fall within the intended scope hereof. Thus, it should be apparent that there has been provided in accordance with the present invention a method and apparatus for an intraoral device that fully satisfies the objectives and advantages set forth above. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. An intraoral device, comprising a pad conformed to fit a palate of a human mouth, the pad including: a first surface configured to lie along the palate of the human mouth; and an exterior surface exposed to the tongue of the human mouth, wherein the exterior surface is at least partially abrasive.

2. The device of claim 1, wherein the pad includes a bio-adhesive on the first surface.

3. The device of claim 2, wherein the bio-adhesive is comprised of at least one of natural gums, synthetic polymers, mucoadhesive polymers, hydrophillic polymers, natural polymers, saccharine derivatives, and cellulose derivatives.

4. The device of claim 2 wherein the bio-adhesive is comprised of at least one of a polyacrylate, polyacrylic acid, a carboxyvinyl polymer, a derivative of a carboxyvinyl polymer, pectin, alginate, karaya gum, pectin gum, xanthan gum, and guar gum.

5. The device of claim 1 wherein a predetermined dosage of medication is embedded in the pad.

6. The device of claim 5 wherein medication comprises a plurality of particles distributed through at least a portion of the pad.

7. The device of claim 6 wherein at least some of the particles have enteric coatings exposed to the tongue of the wearer.

8. The device of claim 5, wherein the medication is comprised of a flavored substance.

9. The device of claim 8, wherein the flavored substance is comprised of at least one of menthol, menthyl lactate, peppermint oil, paramenthane and caboxyamides.

10. The device of claim 5, wherein the medication is comprised of at least one of anesthetic, analgesic, antibiotic, anti-inflammatory, anti-bacterial, antimicrobial, antifungal, antihistamine, steroid and anti-neoplastic agents.

11. The device of claim 1, wherein the exterior surface is comprised of a water-insoluble material.

12. The device of claim 11 wherein the water-insoluble material is comprised of at least one of a silicate, polypropylene, a wax, a natural polymer and a synthetic polymers.

13. The device of claim 1, further comprising at least one self-supporting layer, disposed between the exterior surface and the first surface.

14. The device of claim 13, wherein the self-supporting layer is comprised of at least one of polyester, polypropylene, nylon, rayon, cellulose acetate, cellulose derivatives, cloth, paper, wax, synthetic fibers and natural fibers.

15. The device of claim 14 wherein the self-supporting layer is at least one of a spray-bonded, spun-bonded, carded, thermal bonded, melt-blown, needled, wet-laid and dry-laid layer.

16. The device of claim 1 wherein the pad includes an airborne contaminant absorber and the exterior surface is absorbent.

17. An intraoral device, comprising a pad conformed to fit a palate of a human mouth, the pad including: a first surface configured to lie along the palate of a human mouth; and tongue scraping means for scraping a tongue, wherein the tongue scraping means is on a second surface.

18. The device of claim 17, wherein the first surface includes means for adhering the pad to the palate.

19. The device of claim 17 further compassing means for providing medication, embedded in the pad.

20. The device of claim 19, wherein the means for providing medication includes means for providing a flavored substance.

21. The device of claim 17, further comprising means for supporting the pad, disposed between the first surface and the tongue scraping means.

22. The device of claim 1 wherein the pad includes means for absorbing airborne contaminants disposed within the pad.

23. A method of treating a mouth over a period of time, comprising: forming a pad having a first surface configured to lie along the palate of the human mouth; and providing an exterior surface of the pad that is at least partially abrasive.

24. The method of claim 23, further comprising applying a bio-adhesive on the first surface.

25. The method of claim 24, wherein applying a bio-adhesive includes applying at least one of natural gums, synthetic polymers, mucoadhesive polymers, hydrophillic polymers, natural polymers, saccharine derivatives, and cellulose derivatives, a polyacrylate, polyacrylic acid, a carboxyvinyl polymer, a derivative of a carboxyvinyl polymer, pectin, alginate, karaya gum, pectin gum, xanthan gum, and guar gum.

26. The method of claim 23 further comprising embedding a predetermined dosage of medication in the pad.

27. The method of claim 26 embedding medication comprises embedding a plurality of enteric coated particles through at least a portion of the pad.

28. The method of claim 26, wherein embedding medication comprises embedding a flavored substance.

29. The method of claim 28, wherein embedding a flavored substance comprises embedding at least one of menthol, menthyl lactate, peppermint oil, paramenthane and caboxyamides.

30. The method of claim 26, wherein embedding medication comprises embedding at least one of anesthetic, analgesic, antibiotic, anti-inflammatory, anti-bacterial, antimicrobial, antifungal, antihistamine, steroid and anti-neoplastic agents.

31. The method of claim 23, wherein providing the exterior surface includes providing a surface comprised of a water-insoluble material.

32. The device of claim 23, further comprising providing at least one self-supporting layer, disposed between the exterior surface and the first surface.

33. A method of treating a mouth over a period of time, comprising: placing a pad in the mouth; adhering a first surface to the palate of the mouth; and scraping the tongue against an exterior surface of the pad that is at least partially abrasive.

34. The method of claim 33, wherein adhering includes a bio-adhering the first surface to the palate.

35. The method of claim 34 further comprising releasing over time a predetermined dosage of medication.

36. The method of claim 35, wherein releasing includes releasing a flavored substance.

37. An intraoral device for providing medication to a user, comprising: a pad conformed to fit the palate of a human mouth, the pad including: an airborne contaminant absorber; a first surface configured to lie along the palate of a human mouth, and having a bio-adhesive disposed thereon; an exterior absorbent surface exposed to the tongue of a human mouth; a predetermined dosage of medication embedded in the pad, wherein the predetermined dosage of medication comprises a plurality of particles distributed through at least a portion of the pad; and wherein at least some of the particles have enteric coatings exposed to the tongue of the wearer.

38. The device of claim 37, wherein the bio-adhesive is comprised of at least one of natural gums, synthetic polymers, mucoadhesive polymers, hydrophillic polymers, natural polymers, saccharine derivatives, and cellulose derivatives.

39. The device of claim 37 wherein the bio-adhesive is comprised of at least one of a polyacrylate, polyacrylic acid, a carboxyvinyl polymer, a derivative of a carboxyvinyl polymer, pectin, alginate, karaya gum, pectin gum, xanthan gum, and guar gum.

40. The device of claim 38, wherein the medication is comprised of a flavored substance.

41. The device of claim 40, wherein the flavored substance is comprised of at least one of menthol, menthyl lactate, peppermint oil, paramenthane and caboxyamides.

42. The device of claim 40, wherein the medicine is comprised of at least one of anesthetic, analgesic, antibiotic, anti-inflammatory, anti-bacterial, antimicrobial, antifungal, antihistamine, steroid and anti-neoplastic agents.

43. The device of claim 42, wherein the exterior surface is comprised of an abrasive material.

44. The device of claim 43, wherein the exterior surface is comprised of a water-insoluble material.

45. The device of claim 43 wherein the water-insoluble material is comprised of at least one of a silicate, polypropylene, a wax, a natural polymer and a synthetic polymers.

46. The device of claim 44, further comprising at least one self-supporting layer, disposed between the exterior surface and the first surface.

47. The device of claim 46, wherein the self-supporting layer is comprised of at least one of polyester, polypropylene, nylon, rayon, cellulose acetate, cellulose derivatives, cloth, paper, wax, synthetic fibers and natural fibers.

48. The device of claim 47 wherein the self-supporting layer is at least one of a spray-bonded, spun-bonded, carded, thermal bonded, melt-blown, needled, wet-laid and dry-laid layer.

49. An intraoral device for providing medication to a user, comprising: a pad conformed to fit the palate of a human mouth, the pad including: means for absorbing airborne contaminants; means for bio-adhering a first surface configured of the pad to the palate of a human mouth; and means for applying medication from the pad to the mouth.

50. The device of claim 49, wherein an exterior surface of the pad includes means for scraping the tongue.

51. The device of claim 50, wherein an interior of the pad comprises means for self-supporting the pad.

52. An intraoral device, comprising a pad having; a first surface having a bio-adhesive disposed thereon, and an exterior surface exposed to the tongue of a human mouth, wherein the exterior surface is at least partially abrasive.

53. An intraoral device, comprising a pad conformed to fit a mucosal surface of the human mouth, the pad including: a first surface configured to adhere to a mucosal surface in the human mouth; and an exterior surface exposed to the human tongue, wherein the exterior surface is at least partially abrasive.

54. The device of claim 53 wherein a predetermined dosage of medication is embedded in the pad.

55. The device of claim 54 wherein medication comprises a plurality of particles distributed throughout at least a portion of the pad.

56. The device of claim 55 wherein at least some of the particles have enteric coatings exposed to the tongue of the wearer to delay release of at least some of the particles.

57. An intraoral device, comprising a self-supporting layer having medication, a bio-adhesive, and abrasive components.

58. The device of claim 57, wherein the medication, a bio-adhesive, and abrasive components are not on a surface of the devices.
